(19) United States
(12) Patent Application Publication
(10) Pub. No.: US 2026/0358313 A1
(43) Pub. Date: Nov. 12, 2026

(54) AEROSOL EXPOSURE TESTING CONTAINER

(71) Applicant: The Trustees of Indiana University, Bloomington, IN (US)

(72) Inventors: Melissa Ann Kacena, Zionsville, IN (US); Julian Andres Balanta Melo, Indianapolis, IN (US)

(21) Appl. No.: 19/134,930

(22) PCT Filed: Nov. 28, 2023
(86) PCT No.: PCT/US2023/081300

§ 371 Date: Jun. 2, 2025

Related U.S. Application Data
(60) Provisional application No. 63/432,473, filed on Dec. 14, 2022.

(51) Int. Cl. G01N 33/00

(52) U.S. Cl. CPC G01N 33/0011

(57) ABSTRACT

A modular testing container that can include a plurality of segments, at least one of the segments having an upper seal that can be positioned about the upper periphery and/or a lower seal that can be positioned about a lower periphery of the segment. The modular testing container can also include at least one lock that is adapted to selectively secure two adjacent segments of the plurality of segments. Further, at least one of the upper seal and the lower seal is configured to be at least partially compressed between the two adjacent segments to provide a hermetic seal therebetween when the at least one lock is secured to the two adjacent segments, and the opening at the upper periphery of one of the two adjacent segments is adjacent to the opening at the lower periphery of the other segment of the two adjacent segments.

### REFERENCE TO GOVERNMENT GRANTS

This invention was made with government support under TR002529 awarded by National Institutes of Health. The Government has certain rights in the invention.

### FIELD OF THE DISCLOSURE

The present disclosure generally relates to a testing container for testing airborne transmission of biological hazards. More specifically, the subject disclosure is generally related to an aerosol exposure testing container having a configuration that accommodates passage of individual container segments through an opening of, and subsequent assembly of the container within, an enclosed or enclosable cabinet, or can be used outside of a cabinet.

### BACKGROUND

Occupational risks relating to biological exposure to airborne diseases, among other airborne transmissible hazards, remains an area of interest. For example, in the context of clinical dentistry, and area of research interest is directed to airborne exposure to bacteria, fungi, and viruses that can be present in the oral cavity of a patient. Like other medical and professional fields, current approaches to minimizing airborne transmission of such hazards has included use of personal protection equipment (PPE). Further, with respect to at least dentistry, other approaches have also included use of mouthwashes, extraction devices and virucidal agents. However, such approaches have shown varying degrees of effectiveness. The effectiveness of such approaches can further be adversely impacted as the contagiousness of certain virus strains increases, including, for example, virus strains related to Coronavirus (COVID). Thus, with respect to at least dentistry, as well as otorhinolaryngology, internal medicine, surgery, and anesthesiology, among other fields and disciplines in which individuals can be exposed to airborne biological hazards, there is an interest in developing more effective methods and tools for controlling and mitigating such related occupational hazards.

Often, studies relating to airborne diseases and toxins can be conducted within a biosafety, microbiological safety, or safety cabinet, enclosure or hood, among other similar devices (collectively referred to as “biosafety cabinets”). Such biosafety cabinets are typically designed to provide personal and environmental protection against exposure to biological hazards that may be present within the biosafety cabinet. However, by being configured to confine such hazards, including biohazards, within the interior of the biosafety cabinet, access to the interior of the biosafety cabinet, such as, for example, entry into the interior through an opening of the biosafety cabinet, can be limited. For example, at least certain biosafety cabinets provide a relatively small opening for external access to the interior of the cabinet. Moreover, in some instances, such openings can be approximately 20 centimeters (cm) in height, which can be prohibitive with respect to at least test equipment that can be utilized within the interior of the biosafety cabinet.

### SUMMARY

The present disclosure may comprise one or more of the following features and combinations thereof.

In one embodiment of the present disclosure, a testing container can include a plurality of segments. Each segment of the plurality of segments can comprise a plurality of segment walls that define an upper periphery, a lower periphery, and an interior section of the segment, and an opening at least at one of the upper periphery and the lower periphery, the opening being in fluid with the interior section. At least one segment of the plurality of segments can include an at least one of an upper seal that can be positioned about the upper periphery and/or a lower seal that can be positioned about a lower periphery of the at least one segment. The testing container can also include at least one lock that is adapted to selectively secure two adjacent segments of the plurality of segments. Additionally, at least one of the upper seal and the lower seal is configured to be at least partially compressed between the two adjacent segments when the at least one lock is secured to the two adjacent segments, and the opening at the upper periphery of one of the two adjacent segments is adjacent to the opening at the lower periphery of the other segment of the two adjacent segments.

In another embodiment, the testing container is part of a system that can also include a model that is sized to be positioned within an interior area of the testing container, a container configured to contain a fluid, a fluid introduction device adapted to provide a force to at least deliver the fluid to the model, and an aerosol generating device.

These and other features of the present disclosure will become more apparent from the following description of the illustrative embodiments.

### BRIEF DESCRIPTION OF THE DRAWINGS

The disclosure contained herein is illustrated by way of example and not by way of limitation in the accompanying figures. For simplicity and clarity of illustration, elements illustrated in the figures are not necessarily drawn to scale. For example, the dimensions of some elements may be exaggerated relative to other elements for clarity. Further, where considered appropriate, reference labels have been repeated among the figures to indicate corresponding or analogous elements.

FIG. 1 illustrates an exemplary block representation of an aerosol exposure testing system having a modular testing container positioned within a biosafety cabinet.

FIGS. 2 and 3 illustrate a front side view and a top side view, respectively, of an example of a first, lower segment of the exemplary modular testing container shown in FIG. 1.

FIG. 4 illustrates a front side perspective view of the first, lower segment of the modular testing container shown in FIG. 2 but without an upper seal.

FIG. 5 illustrates a front side perspective view of an exemplary second, upper segment of the modular testing container shown in FIG. 2 but without a lower seal.

FIG. 6 illustrates a front side view of the exemplary second, upper segment of the modular testing container.

FIG. 7 illustrates a front side perspective view of an exemplary third, middle segment of the modular testing container shown in FIG. 2 but without an upper seal and a lower seal.

FIG. 8 illustrates a front side view of the exemplary third, middle segment of the modular testing container.

FIG. 9 illustrates a front view of an exemplary lock body of a lock for the modular testing container.

FIG. 10 illustrates a cross sectional view of the lock body taken along line 10-10 in FIG. 9.

FIG. 11 illustrates a front side view a lock in locking engagement with a pair of adjacent segments of the modular testing container.

Corresponding reference numerals are used to indicate corresponding parts throughout the several views.

### DETAILED DESCRIPTION

While the concepts of the present disclosure are susceptible to various modifications and alternative forms, specific embodiments thereof have been shown by way of example in the drawings and will be described herein in detail. It should be understood, however, that there is no intent to limit the concepts of the present disclosure to the particular forms disclosed, but on the contrary, the intention is to cover all modifications, equivalents, and alternatives consistent with the present disclosure and the appended claims.

References in the specification to “one embodiment,” “an embodiment,” “an illustrative embodiment,” etc., indicate that the embodiment described may include a particular feature, structure, or characteristic, but every embodiment may or may not necessarily include that particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to effect such feature, structure, or characteristic in connection with other embodiments whether or not explicitly described. Additionally, it should be appreciated that items included in a list in the form of “at least one A, B, and C” can mean (A); (B); (C); (A and B); (A and C); (B and C); or (A, B, and C). Similarly, items listed in the form of “at least one of A, B, or C” can mean (A); (B); (C); (A and B); (A and C); (B and C); or (A, B, and C).

In the drawings, some structural or method features may be shown in specific arrangements and/or orderings. However, it should be appreciated that such specific arrangements and/or orderings may not be required. Rather, in some embodiments, such features may be arranged in a different manner and/or order than shown in the illustrative figures. Additionally, the inclusion of a structural or method feature in a particular figure is not meant to imply that such feature is required in all embodiments and, in some embodiments, may not be included or may be combined with other features.

A number of features described below may be illustrated in the drawings in phantom. Depiction of certain features in phantom is intended to convey that those features may be hidden or present in one or more embodiments, while not necessarily present in other embodiments. Additionally, in the one or more embodiments in which those features may be present, illustration of the features in phantom is intended to convey that the features may have location(s) and/or position(s) different from the locations(s) and/or position(s) shown.

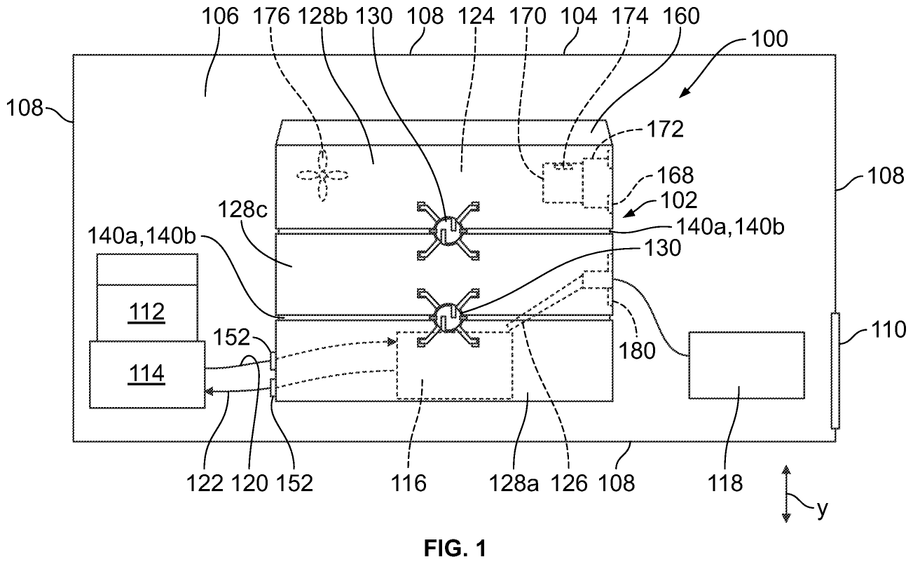

FIG. 1 illustrates an exemplary block representation of an aerosol exposure testing system 100 having a modular testing container 102 positioned within a biosafety cabinet 104. As seen, the illustrated aerosol exposure testing system 100, also referred to herein as the testing system or system 100, can be positioned within an interior area 106 of the biosafety cabinet 104 that may be at least partially, if not completely enclosed by one or more walls 108 of the biosafety cabinet 104. Moreover, access to the interior area 106 of the biosafety cabinet 104 can be generally limited to a selectively openable opening 110 that can be positioned in at least one sidewall 108. However, such an opening 110 is typically relatively small, particularly with respect to a vertical height (as generally indicated by the “y” direction in FIG. 1). For example, often such an opening 110 can have a vertical height of around 20 cm.

According to certain embodiments, the testing system 100 can include the modular testing container 102, a fluid 112, a fluid introduction device 114, a model 116, and an aerosol generating device 118. As discussed below, the modular container 102 is configured to address space limitations that can be encountered in connection with access to the interior area 106 of the biosafety cabinet 104, so that container 102 can be used in a variety of different types of testing and studies, including, but not limited to, testing of different hypothesis relating to physical barriers and surface coatings to avoid exposure contamination by at least microorganisms and/or other potentially harmful exposure, including, but not limited to toxins and chemicals.

The fluid 112 can be correspond to a fluid that can include or contain a biological hazard, such as, for example, a bacteria, fungi, and/or virus. A variety of fluids can be utilized as the fluid 112, including, but not limited to, actual or artificial bodily fluids, among other fluids. For example, according to certain embodiments, the fluid 112 can be actual or artificial saliva or blood, among other fluids, that contain one or more biological hazards. The fluid 112 can also contain a variety of other secondary materials, including, but not limited to other fluids or solid matter, including, but not limited to dyes, such as, a fluorescent dye (e.g., fluorescein, among others). Further, the fluid 112 can be housed within a reservoir or container 102.

The fluid introduction device 114 is configured to provide a force used to transport at least a portion of the fluid 112 from the container 102 to the model 116, and/or from the model 116 to the container 102. Thus, for example, the fluid introduction device 114 may be utilized in connection with an open loop system in which at least a portion of the fluid 112 is delivered via one or more supply lines or conduits 120 from the container 102 to the model 116, but delivered fluid 112 is not necessarily returned from the model 116 to the container 102. Alternatively, the fluid introduction device 114 can be used in connection with closed loop system in which, in addition to delivery of fluid 112 from the container 102 to the model 116 via the one or more supply lines 120, at least a portion of the delivered fluid 112 returns from the model 116 to the container 102 via one or more return lines or conduits 122.

A variety of different types of fluid introduction devices 114 can be utilized by the testing system 100. For example, according to certain embodiments, the fluid introduction device 114 can be one or more pumps, such as a peristaltic pump, among other types of pumps. The fluid introduction device 114 can also be configured to assist in the delivery of the fluid 112 in a variety of different manners. For example, according to certain embodiments, fluid introduction device 114 can be configured for a continuous and/or semi continuous flow of the fluid 112 to the model 116. According to other embodiments, the fluid introduction device 114 can be adapted to provide a sporadic supply of fluid 112 to the model 116.

Additionally, the outlet of the supply line 120 and/or the model 116 can be configured for the dispensing of the fluid 112 onto and/or around the model 116 in a variety of manners. For example, according to certain embodiments, the supply line(s) 120 and/or model 116 can include one or more orifices and/or manifolds that can direct a flow of the liquid in a manner in which the fluid 112 is delivered in a manner in the fluid 112 flows out of the supply line 120 and/or model 116 so as to cascade along at least a portion of the model 116. Alternatively, or additionally, such orifices and/or manifolds can be configured such that the fluid 112 can be sprayed onto, and/or around, the model 116. Further, with such a spraying embodiment, the fluid introduction device 114 can be operated such that fluid 112 can be outputted from the supply line(s) 120 and/or model 116 in a manner that may generally replicate a cough or sneeze.

The model 116 can be positioned within an inner area 124 of the modular testing container 102, and can, according to certain embodiments, be a representation of a device or object from which a biological hazard, chemical, and/or toxin can be transmitted, including, but not limited, via airborne transmission. For example, the model 116 can be a model of a body part, limb, or other feature of an anatomy of a person or animal, among other types of models. As one non-limiting example, the model 116 can be a model of the human mouth that can contain a biohazard, such as, for example, one or more bacteria, fungi, and/or viruses. In such an example, the model 116 can be adapted to provide one or more locations for the presence of the biohazard, including along one or surfaces of the mouth model, such as, for example, a model of a tooth/teeth and/or tissue, among other surfaces, and/or within a liquid that can be present in the mouth model, such as, for example, in the form of a contaminant within artificial saliva.

According to the illustrated embodiment, the aerosol generating device 118 is positioned and configured to facilitate at least a portion of the fluid 112, and/or contents in the fluid 112, such as a biological hazard(s) and/or secondary materials, becoming airborne. According to certain embodiments, the aerosol generating device 118 can be adapted to generate an ultrasonic vibration at or around the model 116. Moreover, the aerosol generating device 118 can be, or can be adapted to generally replicate, a device that, depending on the occupation, may facilitate a biological hazard(s) in a liquid or fluid becoming airborne. Thus, according to certain embodiments, the aerosol generating device 118 can be a tool or instrument utilized in medical situations, including, for example, an ultrasonic scaler, oral suction device, grinder, ultrasonic scalpel, and/or bone saw, among other devices. In the illustrated embodiment, the aerosol generating device 118 is an ultrasonic scaler having a tip 126 positioned to contact, or be in relative close proximity to, the model 116 in a manner that can transfer ultrasonic vibrations to the model 116, and thus to the fluid 112 within, on, and/or around the model 116.

Operation of the aerosol generating device 118, including activation/deactivation, intensity of vibrations, and/or vibration duration, among other settings, can be controlled in a variety of manners. For example, according to certain embodiments, the aerosol generating device 118 is adapted for remote control, and, moreover, for control from an area remote to the interior of the biosafety cabinet 104 in which the aerosol generating device 118 may be positioned.

Referencing FIGS. 1-8, the modular testing container 102 can comprise a plurality of sections or segments 128a-c. The segments 128a-c are configured such that, when each segment 128a-c is positioned in the interior area 106 of the biosafety cabinet 104, the segments 128a-c can be assembled together to form the container 102. In the illustrated embodiment, the segments 128a-c can be assembled together to provide a stacked arrangement of segments 128a-c. Moreover, as seen FIG. 1, the segments 128a-c can be stacked in a generally vertical direction so as to form the container 102. Further, the segments 128a-c can be configured for a locking engagement with each other, or can be adapted to be securely coupled together via a lock 130, as discussed below.

Each segment 128a-c (each generally referred to herein as segment 128) can comprise one or more, if not a plurality of, segment walls 132a-d that can generally define an interior section 134 of the associated segment 128. Additionally, when the plurality of segments 128a-c are assembled together, the segment walls 132a-d can generally define at least a portion of an inner area 124 of the testing container 102. The segment(s) walls 132a-d can be configured to provide the segment 128 with a variety of cross sectional shapes. For example, as seen in at least FIGS. 3-5, each segment 128 can, according to certain embodiments, include four segment walls 132a-d that are arranged to provide a square or rectangular cross sectional shape. However, fewer or more the four segment walls 132a-d can be utilized, including in connection with providing the segments 128a-c with a variety of other circular and non-circular shapes, including, for example, triangular, trapezoidal, polygonal, round, and oval shapes, among other shapes.

The segment walls 132a-d can be constructed from a variety of materials, including, but not limited to, an acrylic material. Further, according to certain embodiments, one or more segment walls 132a-d of one or more of the segments 128a-c is generally transparent so as to accommodate visual access to the inner area 124 from a location at least outside of the testing container 102. According to certain embodiments, each segment wall 132a-d for one or more of the segments 128a-c may be constructed from a material that may be similar to the material used for the other segment walls 132a-d. Additionally, one or more segments 128a-c can have one or more segment walls 132a-c that is/are constructed form a material that may be different than a material used for the construction of one or more segment walls 132a-d of at least another segment 128a-c. According to the illustrated embodiment, the segment wall 132a-d of each segment 128a-c is constructed from the same type of material.

At least a portion of each segment wall 132a-d can include an upper surface 142 and a lower surface 144. The upper surface 142 of the segment walls 132a-d can generally define the upper periphery 136 of the associated segment 128, and the lower surface 144 of the segment walls 132a-d can generally define a lower periphery 138 of the associated segment 128. Further, according to certain embodiments, the upper periphery 136 and/or the lower periphery 138 of one or more segments 128a-c can include an upper hermetic seal 140a and/or a lower hermetic seal 140b that is/are adapted for a sealing engagement with the adjacent upper or lower seal 140a, 140b of another segment 128a-c and/or an adjacent surface of a segment wall 132a-d of another segment 128a-c. Moreover, the upper and lower seal(s) 140a, 140b can be adapted to prevent and/or minimize the ingress and egress of air or liquids in an area between neighboring segments 128a-c. The hermetic upper and lower seals 140a, 140b, which can generally be referred to herein as upper seal 140a or lower seal 140b, can be constructed form a variety of materials used for providing seals, including, but not limited to, silicon.

The distance between the upper periphery 136 (or any upper seal 140a along the upper periphery 136) and the lower periphery 138 (or any lower seal 140b along the lower periphery 138) for each individual segment 128 can be less than the height of the opening 110 of the biosafety cabinet 104 so as to accommodate passage of the segment 128, and any associated upper seal 140a and/or lower seal 140b) through the opening 110 of the biosafety cabinet 104. Thus, for example, for applications with biosafety cabinets 104 having an opening 110 having a height of 20 cm, the corresponding height of each segment 128, such as, for example, between the upper and lower peripheries 136, 138 and/or between any such seal(s) 140a, 140b on the upper and lower peripheries 136, 138, can be less than 20 cm, including, for example 15-18 cm, and, more specifically, 17 cm, among other heights. Further, the height of each segment 128a-c may or may not be similar to the height of the other segments 128a-c.

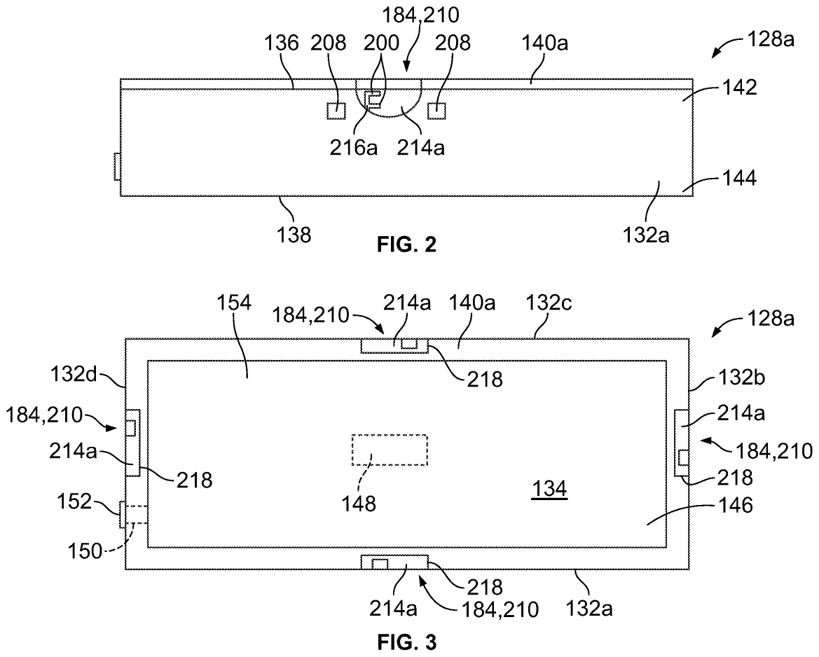

FIGS. 2 and 3 illustrate a front side view and a top side view, respectively, of an example of a first, lower segment 128a of the exemplary modular testing container 102. In addition to the segment walls 132a-d, the lower segment 128a can include a base wall 146 that can extend at or around the lower periphery 138, as well as between, the segment walls 132a-d of the lower segment 128a. Moreover, the base wall 146 can provide a bottom or base wall 146 for the container 102. Thus, for example, the base wall 146 can define at least a portion of the inner area 124 of the container 102, and provide a surface upon which the model 116 can be placed and/or selectively secured. For example, according to certain embodiments, the base wall 146 can include, or be coupled to, a bracket, or include an area for mechanical attachment of the model 116 to the base wall 146. Such attachment can also be configured to accommodate selective detachment of the model 116, or a portion of the model 116, from the lower segment 128a. Optionally, according to certain embodiments, the base wall 146 can include a magnet 148 that can be utilized to interact with another magnet that is coupled to the model 116, or to interact with a metallic surface of the model 116, so as to at least assist in retaining the model 116 at a selected position in the container 102 and/or along the base wall 146.

According to the illustrated embodiment, a segment wall 132d of the lower segment 128a can include one or more orifices 150 to accommodate passage of one or more supply lines 120 and/or return lines 122 through the segment wall 132d and into the inner area 124 of the container 102. Further, the orifice(s) 150 can be sized for mating engagement of a conduit seal 152 that can assist in creating a seal between the supply line(s) 120 and/or return line(s) 122 and the segment wall 132d that can prevent or minimize the ingress/egress of air and/or fluid into/from the inner area 124 of the container 102. Thus, for example, the conduit seal 152 can be configured to hermetically seal the orifice 150. Although illustrated in FIG. 3 as being positioned outside of the inner area 124 of the container 102, the conduit seal 152 can be positioned at a variety of locations, including inside or outside of the container 102 as well as at least partially within the conduit orifice 150, as well as combinations thereof.

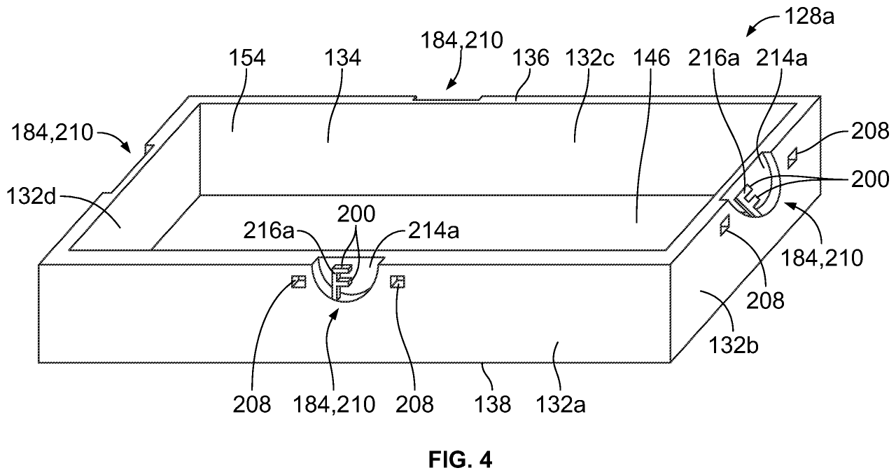

The portion of the lower segment 128a around the upper periphery 136, and moreover between the segment walls 132a-d around the upper periphery 136, can be generally open such that, when the lower segment 128a is assembled with at least one other segment 128b, 128c, the interior sections 134 of the lower segment 128a and the other segments 128b, 128c can be in fluid communication with each other. Thus, the upper periphery 136 of the lower segment 128a can generally define an opening 154 into the interior section 134 of the lower segment 128a. Further, according to the illustrated embodiment, the lower segment 128a can include an upper seal 140a that extends around at least a portion of the upper periphery 136 of the lower segment 128a, and which is configured to abut a lower seal 140b and/or lower periphery 138 of another, adjacent segment 128b, 128c. Alternatively, as seen in FIG. 4, according to another embodiment, the lower segment 128a may not include an upper seal 140a, and instead the upper periphery 136 of the lower segment 128a can be abutted by a lower seal 140b that is positioned on the lower periphery 138 of the other, adjacent segment 128b, 128c. According to certain embodiments, the lower periphery 138 of the lower segment 128a is configured to abut a surface of the biosafety cabinet 104 or other surface beneath the container 102, and thus the lower periphery 138 of the lower segment 128a may not include, or be coupled to, a lower seal 140b.

A seen in at least FIGS. 1, 5, and 6, the container 102 can also include at least a second, upper segment 128b that can, when the container 102 is assembled, be at a vertical position that is above at least the lower segment 128a and a middle segment(s) 128c, if any. Moreover, according to certain embodiments, the upper segment 128b can be adapted to be at the top of the container 102, and/or at a vertical position that is higher than the vertical position(s) of the other segments 128a, 128c of the container 102.

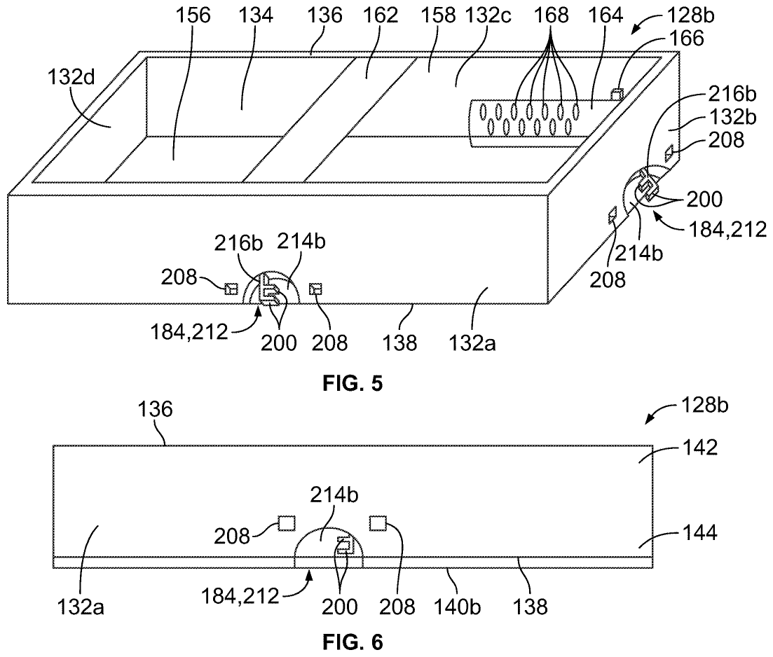

The lower periphery 138 of the upper segment 128b can generally define an opening 156 into the upper segment 128b such that the interior section 134 of the upper segment 128b can generally be in fluid communication with at least the interior section 134 of at least the lower segment 128a when the container 102 is assembled. Further, the lower periphery 138 of the upper segment 128b can include a lower seal 140b, as seen in FIG. 6, or may not include any upper and lower seal 140a, 140b, as seen in FIG. 5.

According to certain embodiments, the upper periphery 136 of the upper segment 128b can also generally define an opening 158 to the interior section 134 of the upper segment 128, and/or to the inner area 124 of the container 102. Further, the upper periphery 136, or other portions of the container 102, can be coupled to, and/or be configured to receive removable placement of, a filter 160 that can be positioned at least adjacent to the opening 158 of the upper segment 128b and/or of the container 102. According to certain embodiments, the filter 160 can be a HEPA filter. Thus, as seen in at least FIG. 5, according to certain embodiments, the upper segment 128b can include a support structure 162, such as, for example, one more side and/or cross beams and/or recesses, among other support structures, at or around the upper periphery 136 that can provide at least some structural support for placement of the filter 160 on the container 102. Alternatively, according to other embodiments, rather than having an opening 158 at the upper periphery 136 of the upper segment 128b, the upper segment 128b can be enclosed at or around the upper periphery 136 by an upper wall.

Referencing FIGS. 1 and 5, according to certain embodiments, one or more segments 128a-c of the container 102 can also include an animal restraint platform 164 that can house one or animals. Moreover, the restraint platform 164 can be configured to house, as well as confine, one or more animals within an interior of the restraint platform 164, and thus within the inner area 124 of the container 102. The restraint platform 164 can therefore be configured for use with animals that may be part of a study or testing being conducted using the container 102.

The restraint platform 164 can be secured to a segment 128a-c in a variety of manners. For example, according to certain embodiments, the restraint platform 164 can be selectively removably coupled to a segment 128a-c, such as, for example, the upper segment 128b, via support bracket 166 that can be attached to an adjacent segment wall 132b. Such a configuration can accommodate, while the restraint platform 164 is detached from the associated segment 128b, placement of animal(s) within an opening in the restraint platform 164, before the restraint platform 164 is subsequently securely coupled to the segment 128b via use of the support bracket 166. According to certain embodiments, an opening for receiving placement of the animal in the platform 164 can be located at an end of the restraint platform 164 such that, when the restraint platform 164 is secured to the segment 128b, the opening is adjacent to the segment wall 132b so as to prevent egress of the animal(s) from the interior of the restraint platform 164.

The restraint platform 164 can have a variety of configurations, including embodiments in which the platform may, or may not, be partially or completely enclosed. For example, according to certain embodiments, the restraint platform 164 can be an at least partially enclosed tunnel, while according to other embodiments, the restraint platform 164 may be a tray or walkway. Further, for example, according to certain embodiments, the restraint platform 164 can include a plurality of perforations 168 that can facilitate a flow of air, and, moreover, an aerosol containing an airborne hazard, such as, for example, a biohazard(s), chemical, or toxin, into the interior of the restraint platform 164. According to certain embodiments, such plurality of perforations 168 may not be present at a base of the restraint platform 164 so as to not interfere with movement of the animal, including at areas at which a foot or leg of the animal could otherwise pass through at least one of the perforations 168.

According to certain embodiments, the restraint platform 164 can comprise a plurality sections 170, 172 (FIG. 1), at least one section 170 being telescopically coupled to another section 172. Such a telescopic arrangement of the sections 170, 172 can accommodate adjustments in the linear distance at which the restraint platform 164 can extend away from the adjacent segment wall 132b to which the restraint platform 164 is coupled. Moreover, such a configuration can accommodate adjustments in the distance to which the platform 164 can extend into the inner area 124 of the container 102. Thus, the extent the restraint platform 164 is positioned directly over, or horizontally offset from, the model 116 can be adjustable. Additionally, according to certain embodiments, at least one section 170 can be coupled to a magnet 174 (FIG. 1). According to such an embodiment, a tool having a corresponding magnet or magnetic surface can be utilized to interact with the magnet 174 of the restraint platform 164 in a manner that can provide means for adjusting a linear position of that section 170 relative to the other section 172 and/or the adjacent segment wall 132b.

As indicated in FIG. 1, according to certain embodiments, one or more segments 128a-c, such as, for example, the upper segment 128b, can be coupled to a fan or impeller 176 that can facilitate a movement of air, as well as airborne contaminant, such as a biohazard, chemical, or toxin contained therein, in the inner area 124 of the container 102. Such a fan 176 can be powered in a variety of manners, including, for example, via battery and/or light, among other manners of powering the fan 176.

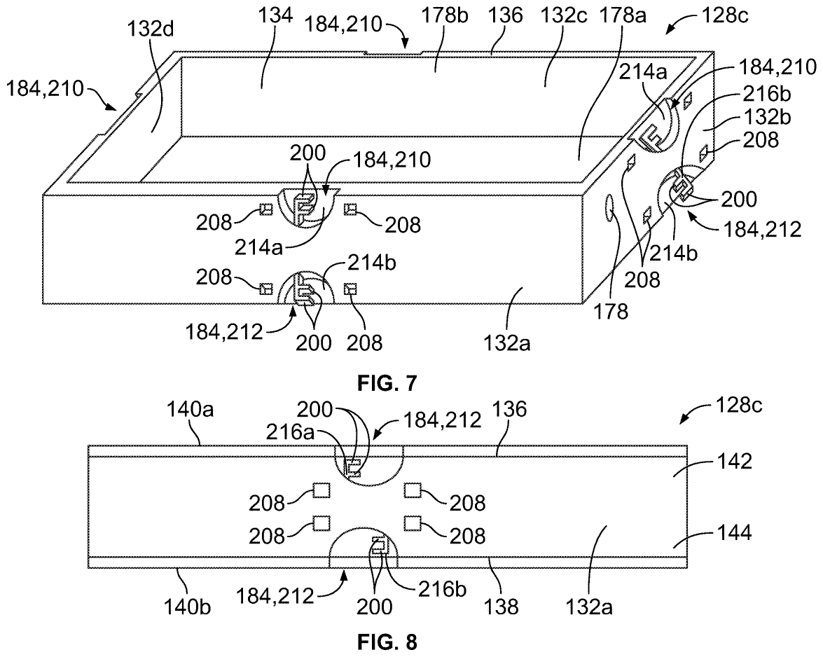

According to certain embodiments, one or more third, or middle segments 128c can be positioned between the lower and upper segments 128a, 128b. Further, the upper and lower peripheries 136, 138 of the middle segment 128c can each define an opening 178a, 178b to the interior section 134 of the middle segment 128c so that the interior sections 134 of each the lower, upper, and middle segments 128a-c are in fluid communication with each other. Further, as indicated by FIGS. 7 and 8, according to various embodiments, the middle segments 128c can include both an upper seal 140a and a lower seal 140b, only one of an upper seal 140a and a lower seal 140b, or have neither an upper seal 140a nor a lower seal 140b.

FIG. 7 also illustrates the middle segment 128c having a generator orifice 178 that can accommodate passage of at least a portion of the aerosol generating device 118, such as, for example, the tip 126 of an ultrasonic scaler, into the inner area 124 of the container 102, among other components of other aerosol generating devices. Similar to the orifice 150 of the lower segment 128a, a seal can be utilized to hermetically seal the generator orifice 178 so as to prevent, and/or limit ingress/egress of air to/from the inner area 124 of the container 102 through the generator orifice 178. Additionally, or alternatively, the middle segment 128c can include an adjustable support 180 (FIG. 1) that can provide an entrance to the generator orifice 178 and/or from the generator orifice 178 into the inner area 124 of the container 102 and/or into the interior section 134 of at least the middle segment 128c. The adjustable support 180 can be adapted to be positioned about the tip 126 of an ultrasonic scaler, or other components of other aerosol generating devices. Additionally, the adjustable support 180 can be hermetically sealed to avoid the escape of contaminated particles from the inner area 124 of the container 102. Further, while the generator orifice 178 is illustrated as extending through the middle segment 128c, the generator orifice 178 could be positioned in another segment 128a, 128b.

As previously mentioned, each segment 128a-c can be secured to at least another segment 128a-c. For example, referencing at least FIGS. 1, 9, and 10, in the illustrated embodiment, the container 102 can include a plurality of locks 130, each lock 130 comprising a lock body 182 and a lock member 184. According to at least certain embodiments, at least a portion of the lock body 182 can be displaceable relative to, and/or about, a least a portion of the lock member 184 so as to lockingly engage the lock body 182 to the lock member 184 in a manner that can securely couple at least a portion of one segment 128a-c to at least a portion of another segment 128a-c via the lock 130.

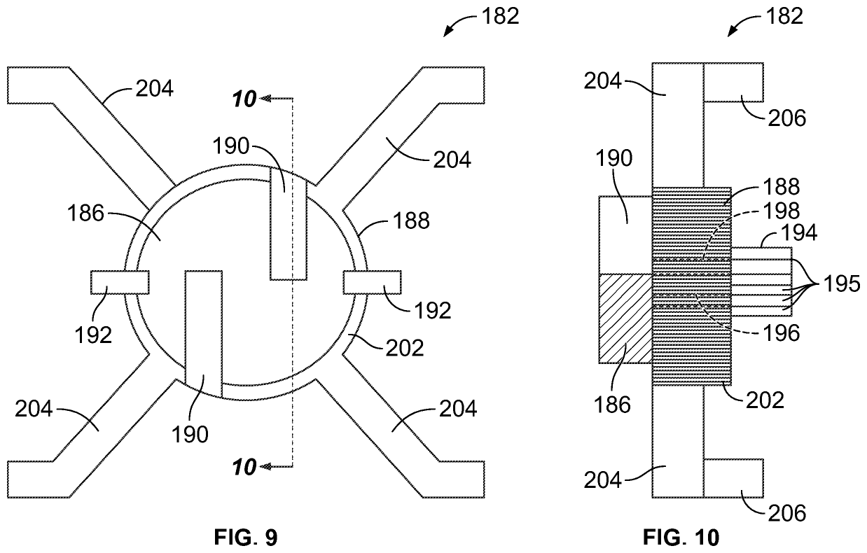

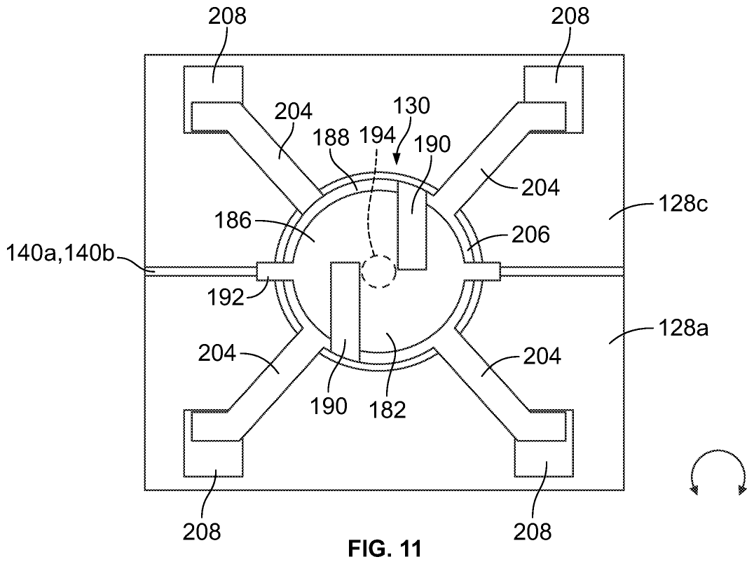

According to certain embodiments, the lock 130 can be configured to provide a twist lock system wherein at least a portion of the lock body 182 can be twisted or rotated about at least a portion of the lock member 184. Referencing FIGS. 9 and 10, according to the illustrated embodiment, the lock body 182 can include a first body 186 and a second body 188, the first body 186 being rotatably displaceable about the second body 188. The first body 186 can be keyed, such as, for example, having one or more cavities 190 that can provide an area or surface at which the first body 186 can be engaged by a tool or digit of an operator in manner that can impart a force to rotate the first body 186. Additionally, the first body 186 can include one or more projections 192 that can also provide areas for engagement with a tool or the operator in connection with imparting a force to rotate the first body 186.

The first body 186 can also be coupled to a pinion 194. In the illustrated embodiment, the first body 186 is coupled to the pinion 194 via a shaft 196 that can extend through an aperture 198 in the second body 188. Accordingly to such an embodiment, rotation of the first body 186 can be translated to the shaft 196 and pinion 194 such that the shaft 196 and pinion 194 are similarly rotatably displaced. The pinion 194 can include a plurality of surface features 195, such as, for example serrations or gear teeth, among other types of surface features, that can matingly engage corresponding protrusions or teeth 200 (collectively referred to as protrusions) of the lock member 184. Additionally, according to certain embodiments, the first body 186 can be coupled to a biasing element, such as, for example, a spring, that can provide a biasing force against, and/or which is transmitted to, the pinion 194.

The second body 188 can be configured to at least assist in positioning the lock 130 relative to two adjacent segments 128a-c. According to the illustrated embodiment, the second body 188 includes a hub 202 and a plurality of arms 204. The hub 200 can be positioned adjacent to the first body 186, and provide the aperture 198 through which the shaft 196 and/or a portion of one or both of the first body 186 and pinion 194 can extend. The plurality of arms 204 can outwardly extend from the hub 202. Each arm 204 can include a projection 206 that can extend generally outwardly from a rear side of the corresponding arm 204. Each projection 206 is positioned to be received in a mating cavity 208 in a segment 128a-c. Thus, according to the illustrated embodiment, as seen in at least FIG. 1, at least one arm 204, and the associated projection 206, is configured such that the projection 206 is received in one segment 128a-c, and at least another one arm 204, and the associated projection 206, is configured such that the projection 206 is received in another, different segment 128a-c.

The lock member 184 can comprise a pair of members, a first member 210 of the pair of members being coupled to, or integrated into, a segment wall 132a-d of a segment 128a-c, and the second member 212 of the pair being coupled to, or integrated into a segment wall 132a-d of another, different segment 128a-c. Further, the first and second members 210, 212 can be positioned along the corresponding segment 128a-c, such that when the container 102 is being assembled, the first member 210 of one segment 128a-c will be generally aligned with the second member 212 of another, adjacent segment 128a-c.

Thus, for example, in the illustrated embodiment, the lower segment 128a can have one or more first members 210 positioned around the upper periphery 136 of the lower segment 128a, and moreover, in one or more of segment walls 132a-d. If the middle segment 128c is to be positioned adjacent to the lower segment 128a, the middle segment 128c can have one or more second members 212 positioned around the lower periphery 138 of the middle segment 128c, and moreover, in one or more of segment walls 132a-d of the middle segment 128c. According to such an embodiment, the second members 212 of the middle segment 128c can be positioned to generally align with the first members 210 of the lower segment 128a when the container 102 is being assembled.

The first member 210 can include a chamber 214a that can extend through the upper periphery 136 of the segment 128a-c. Additionally, the first member 210 can include a rack 216a that is positioned in the chamber 214a, and which includes the plurality of the projections 200. The second member 212 can have a construction generally similar to the first member 210, but rather than extending through the upper periphery 136, the chamber 214b of the second member 212 can instead extend through the lower periphery 138 of the segment 128a-c. Further, as seen in at least FIG. 8, the rack 216b of the second member 212 can be on a side of the chamber 214b of the second member 212 that is opposite to the side at which the rack 216a is at in the chamber 214a of the first member 210. Moreover, the rack 216a of the first member 210 can be positioned to be on a side of the pinion 194 opposite of the side of the pinion 194 to which the rack 216b of the second member 212 is positioned. Additionally, the chambers 214a, 214b of the first and second members 210, 212 can be sized such that when the first and second members 210, 212 are aligned, such as, for example, when the container 102 is being assembled in interior area 106 of the biosafety cabinet 104, the aligned chambers 214a, 214b can receive a portion of the second body 188 of the lock body 182, such as, for example, a rear portion of the second body 188.

According to certain embodiments, the portion of the upper and lower seals 140a, 140b along the upper and/or lower peripheries 136, 138 of the segments 128a-c can have recesses 218 to extend around the profile of the chambers 214a, 214b of the first and second members 210, 212 along the corresponding upper periphery 136 or lower periphery 138. Moreover, such recesses 218 in the seals 140a, 140b can be configured to prevent the seals 140a, 140b from interfering with the lock 130, including the pinion 194 and/or portion of the second body 188, from being received within the chambers 214a, 214b.

As seen in at least FIG. 4, according to the illustrated embodiment, the lower segment 128a can have a plurality of first members 210, including, for example, a first member 210 positioned about each segment wall 132a-d. According to embodiments in which the upper segment 128b is to be positioned adjacent to the lower segment 128a, the upper segment 128b can have a plurality of second members 212 positioned to align with the first members 210 of the lower segment 128. Thus, the upper segment 128b can have a number of second members 212 that may, or may not, be equal to the number of first members 210 of the lower segment 128a. Alternatively, according to embodiments, in which the container 102 is to include at least one middle segment 128c, the middle segment 128c can have a plurality of the second members 212 positioned to align with the first members 210 of the lower segment 128c (or align with first members 210 in an another, adjacent middle section 128c), as well as a plurality of first members 210 positioned to align with the second members 212 of the upper segment 128c (or align with second members 212 in an another, adjacent middle section 128c). Moreover, the middle segment 128c can have a combination of first and second members 210, 212 that are positioned or be aligned with the first and/or second members 210, 212 of another, adjacent middle segment 128c, lower segment 128a, and/or upper segment 128b.

During assembly of a container 102 that includes the lower segment 128a, upper segment 128b, and one middle segment 128c, the middle segment 128c can be positioned on the lower segment 128a such that the first member(s) 210 of the lower segment 128 is/are aligned with the corresponding second member(s) 212 of the middle segment 128c. The lock body 182 can then be positioned such that the projection 206 of each arm 204 is received in a corresponding cavity 208 of the lower segment 128c and the middle segment 128c. Such positioning of the lock body 182 can also include positioning at least the pinion 194 in the aligned chambers 214a, 214b of the first and second members 210, 212 so that the pinion 194 engages the protrusions 200 of the racks 216a, 216b. The first body 186 can be rotated such that the pinion 194 pulls the racks 216a, 216b in a direction that brings the lower periphery 138 of the middle segment 128 into closer proximity to the upper periphery 136 of the lower segment 128c. Thus, for example, rotation of the pinion 194 can facilitate and generally upwardly directed force being exerted against the rack 216a of first member 210 (and thus against at least a portion of the associated segment wall 132a-d of the lower segment 128a), and an opposing downward force being exerted against the rack 216b of second member 212 (and thus against at least a portion of the associated segment wall 132a-d of the middle segment 128c). Such opposing forces can cause the lower and middle segments 128a, 128c being drawn together to secure the lower segment 128a to the middle segment 128c, as well as compress the seal(s) 140a, 140b that can be positioned therebetween. A biasing element or other mechanical feature, such as, for example, a pawl, can be incorporated to retain the pinion 194, and thus racks 216a, 216b at positions that can maintain the force(s) being used to pull the adjacent segments 128a, 128c toward each other.

Similar processes can be repeated using the other locks 130 that can be positioned around the peripheries of the lower and middle segments 128. Further, a similar process can be performed to secure the middle segment 128c to the upper segment 128b. If needed, the filter 160 can also be secured to the upper segment 128a.

### EXAMPLE

A test can be conducted using the assembled container 102 in an interior area 106 of a biosafety cabinet 104 to evaluate the potential for an SARS-COV-2 infection via dental aerosol exposure. According to such an experiment, the container 102 can serve as a reservoir for (1) a portion of an aerosol generating device 118, and more specifically, a tip 126 of an ultrasonic scaler, (2) a model 116 in the form of a teeth model having simulated soft tissue, and which is adapted to accommodate continuous flow of a fluid 112 in the form of a virus-loaded artificial saliva, and a restraint platform 164 configured to house a transgenic mouse. Moreover, such a system can be configured to expose the transgenic mice, in vivo, to dental aerosols generated during ultrasonic scaling in the model 116 of teeth and saliva containing SARS-COV-2, among other virus strains.

Using such a system, dental aerosol particles can be generated using the ultrasonic scaler on the teeth model. The simulated soft tissue of the model can be connected to a fluid introduction device 114, such as, for example, a peristaltic pump, that can serve as a mouth-depicting reservoir for continuous flow of fluid 112 in the form of artificial saliva. The fluid 112 can, for example, be delivered to the model 116 via operation of the fluid introduction device 114 at a defined rate, such as, for example, a rate of 0.5 ml/min. After a predetermined time period, such as, for example, 10 minutes, the saliva-targeted surfaces of the model 116 can be determined using ultraviolet (UV) dark light, which can be included in the interior area 106 of the biosafety cabinet 104.

For dynamic analysis, a six-channel particle counter, which can be located at the level of the mouse restraint platform 164, can be used to quantitatively characterize the dental aerosol particles between 0.3-10 μm. Additionally, after generating the dental aerosol particles during the time period (e.g. 10 minutes), the particle counter can remain operative to determine the settlement time of the dental aerosol particles. The distance between the source of the dental aerosol (teeth model) and the particle counter (e.g., 40 cm) can based on the area of highest contamination in clinical dental settings using ultrasonic scaler devices.

Vivo testing can proceed once dental aerosol particles have been characterized. Thus, while restrained in the restraint platform 164 in the inner area 124 of the container 102. Mice can be exposed to dental aerosol generated by ultrasonic scaling with either a control vehicle or SARS-COV-2 aerosol experimental group, during 10 consecutive minutes. Previous calculation of settlement time for dental aerosol can determine the time after finishing the experiment to open the container 102. The mouse/mice is then returned to its/their original cage(s) for housing. The inside and outside of the container 102 and all equipment can then be cleaned with a cleaner, including, for example, via use of 70% ethanol, among other cleaners, with at least a 10-minute contact time to kill any virus. The container 102 and equipment can then be left to air dried before the container 102 is subsequently set up for the next round of testing. The successful development of this research tool can generate for the first time an in vivo model suitable to test novel strategies, methods, and materials with further research studies, providing direct evidence of risk infection with viruses in the dental aerosol.

While the disclosure has been illustrated and described in detail in the foregoing drawings and description, the same is to be considered as exemplary and not restrictive in character, it being understood that only illustrative embodiments thereof have been shown and described and that all changes and modifications that come within the spirit of the disclosure are desired to be protected.

1. A modular testing container comprising:
a plurality of segments, each segment of the plurality of segments comprising:
a plurality of segment walls that define an upper periphery, a lower periphery, and an interior section of the segment; and
an opening at least at one of the upper periphery and the lower periphery, the opening being in fluid communication with the interior section;
at least one segment of the plurality of segments having at least one of an upper seal positioned about the upper periphery and a lower seal positioned about a lower periphery of the at least one segment; and
at least one lock adapted to selectively couple a pair of adjacent segments of the plurality of segments,
wherein at least one of the upper seal and the lower seal is configured to be at least partially compressed between the pair of adjacent segments when the at least one lock is secured to the pair of adjacent segments, and
wherein the opening at the upper periphery of one adjacent segment of the pair of adjacent segments is adjacent to the opening at the lower periphery of the other adjacent segment of the two pair of adjacent segments.

2. The modular testing container of claim 1, wherein the plurality of segment walls have a vertical height between the upper periphery and the lower periphery that is not greater than 20 centimeters.

3. The modular testing container of claim 1, further including a restraint platform selectively attachable to at least one of the plurality of segments.

4. The modular testing container of claim 3, wherein the restraint platform comprises a plurality of protrusions positioned between opposing ends of the restraint platform.

5. The modular testing container of claim 3, wherein the restraint platform includes a plurality of sections that are telescopically arranged.

6. (canceled)

6. The modular testing container of claim 1, wherein at least one of the plurality of segment walls includes at least one orifice that extends through at least one of the plurality of segment walls.

7. The modular testing container of claim 6, further including at least one seal adapted to be positioned at least partially around the at least one orifice to form a hermetic seal.

8. The modular testing container of claim 1, wherein the plurality of segment walls are arranged to be assembled in a vertically oriented stack.

9. The modular testing container of claim 8, further including a filter.

10. The modular testing container of claim 9, wherein the filter is coupled to the segment of the plurality of segments.

11. The modular testing container of claim wherein the filter is positioned at a top area of the vertically oriented stack.

12. The modular testing container of claim 8, wherein at least one segment of the plurality of segments includes am an upper wall.

13. The modular testing container of claim 1, wherein at least one segment includes a base wall.

14. The modular testing container of claim 13, wherein the base wall is coupled to a magnet.

15. The modular testing container of claim 1, wherein the lock comprises a lock body and a lock member.

16. The modular testing container of claim 15, wherein the lock body comprises a first body and a second body, at least a portion of the first body being rotatable relative to the second body.

17. The modular testing container of claim 16, wherein the first body includes one or more cavities that extend into the first body and provide an area or surface at which the first body is engageable by a tool or a digit in manner that can impart a force to rotate the first body.

18. The modular testing container of claim 16, wherein the lock member includes a first member and a second member, the first member being positioned adjacent to the upper periphery of at least one segment of the plurality of segments, the second member being positioned around the lower periphery of at least one other segment of the plurality of segments.

19. The modular testing container of claim 18, wherein the first and second members each include a rack that is housed in a cavity in at least one segment wall of the plurality of segment walls.

20. The modular testing container of claim 19, wherein the first body is coupled to a pinion that is configured to matingly engage the rack such that a rotation of the pinion by a rotation of the first body transmits a force that displaces the rack.

21.-33. (canceled)
